(19) United States
(12) Patent Application Publication
(10) Pub. No.: US 2025/0120591 A1
(43) Pub. Date: Apr. 17, 2025

(54) DEVICE AND METHOD FOR DETECTING BIOFILM IN THE ORAL CAVITY

(71) Applicant: epitome GmbH, Wien (AT)

(72) Inventors: Andrei SHMELEV, Wien (AT); Bernhard RIEDER, Wien (AT); Bernhard Moritz PICHLER, Wien (AT)

(21) Appl. No.: 18/834,142

(22) PCT Filed: Dec. 21, 2022
(86) PCT No.: PCT/EP2022/087350

§ 371 Date: Jul. 29, 2024

(30) Foreign Application Priority Data
Jan. 28, 2022 (DE) 10 2022 102 045.2

(51) Int. Cl. A61B 5/00; G06T 7/00; G06T 7/10; G16H 30/40

(52) U.S. Cl. CPC A61B 5/0088; A61B 5/0062; A61B 5/0084; G06T 7/0012; G06T 7/10; G16H 30/40; A61B 2560/0406; A61B 2560/0462; A61B 2562/227; G06T 2207/20081; G06T 2207/30036

(57) ABSTRACT

The invention relates to a device for optically detecting biofilm in the oral cavity, wherein at least one sensor unit is provided, wherein the sensor unit has a camera, which points with its optical detection region into a receiving space of the device and serves to record surfaces that are to be detected.

The invention relates to a device and method for detecting biofilm in the oral cavity.

Biofilm in the oral cavity and plaque in particular is a widespread, well-known problem. There are now countless methods and devices intended for removing plaque.

In addition to manual toothbrushes with special bristle profiles, these also include electric toothbrushes with oscillating or vibrating bristle heads. A wide variety of oral irrigators are also known, which use injected liquids to remove plaque. With toothbrushes, there is almost no limit to the number of errors that can be made by the user. The performance of the well-known oral irrigators had to be significantly reduced compared to the old designs, as too strong a jet of liquid caused considerable damage to the gums. The oral irrigators that do not cause damage, however, also do not achieve real cleaning.

It is also known to use intraoral camera units to record optically detectable conditions in the oral cavity. But intraoral camera unit designs that are currently available on the market are too large to be used easily, especially for end-users.

It is also known to illuminate teeth with UV/blue (˜405 nm) light to check for early cavities, cracks in the tooth, and secondary cavities. The contrast medium fluorescein has a fluorescence with the strongest intensity at em=520 to 530 nm when it is excited with light ex=465 to 490 nm. The very narrow wavelength spectrum between the exciting light and the emitted light results in a signal-to-noise ratio that is too low to ensure reliable detection with standard intraoral camera units. With intraoral cameras that are currently on the market, reflections caused by focused lighting and blurred images from the camera unit are currently compensated for only by increasing the distance from the tooth to the sensor unit.

The object of the invention is to detect biofilm efficiently and in a space-saving manner in order to make parallel or subsequent cleaning more effective.

This object is attained by a device with the features of claim 1.

Advantageous modifications are disclosed in the dependent claims.

Another object of the invention is to create a method for detecting biofilm.

This object is attained by a method with the features of claim 31.

Advantageous modifications are disclosed in the dependent claims thereof.

According to the invention, biofilm (plaque) is first made optically detectable and then detected by location and intensity (quantity). The corresponding data, consisting of the coordinates in the oral cavity and, if applicable, an intensity/amount/layer thickness, can be
a) stored and transferred to a cleaning device to enable targeted cleaning;
b) stored and compared to previous or subsequent detections in order to document cleaning progress;
c) stored and made available for therapeutic purposes, in particular for establishing case histories and conducting analyses.

According to the invention, a sensor unit of a biofilm visualization device is proposed, which, after insertion into the oral cavity, is guided over the teeth and thereby detects biofilm with the aid of a contrast medium and transmits the exact position and, if applicable, layer thickness, etc. to the device.

For this purpose, the device consists of a handpiece, which can have a display on the end remote from the user. On the end close to the user, there is a mouthpiece that is inserted into the oral cavity and guides the sensor unit over the teeth.

According to the method, the teeth are stained with a fluorescein-based dye before the detection.

The mouthpiece has at least one camera unit with a camera.

The camera unit alone, for example, has the dimensions 1×1×2.7 mm and the entire sensor unit, including a protective glass, PCB, filter, and camera holder, has a diameter of 8 mm and a height of 3.8 mm. With these dimensions, the unit can be easily inserted into an oral cavity.

To ensure a good signal-to-noise ratio, an optical long-pass filter, which ideally has a cut-off wavelength of 480 nm to 530 nm and in particular around 510 nm and cuts off signals below this, is preferably placed directly in front of the camera. Alternatively or additionally, a band-pass filter or short-pass filter can be placed in front of the LEDs that illuminate the area to be detected in order to limit/focus the wavelength spectrum of the LEDs.

In order to reduce reflections, which can negatively influence the recording and evaluation of the biofilm, a diffuser, particularly in the form of a diffuser foil, can be provided between the PCB and the protective glass.

A polarizer, in particular a circular polarizer, can also be positioned in front of the LEDs and camera in order to minimize reflections.

The above-mentioned measures of providing a long-pass filter, a short-pass filter, a diffuser, and a polarizer can be implemented individually or in any combination with one another.

The device itself can also have biometric security functions. For example, a built-in camera can be used for facial recognition or a fingerprint sensor can be used for fingerprint recognition. The dental layout can also be detected using corresponding optical or mechanical pressure sensors.

Mechanical pressure sensors, for example, are a sensor field that is positioned in a mouthpiece in such a way that it points toward the crowns of the teeth when in use. In addition, the resolution—and therefore the number of sensors in the sensor field—is selected so that a very precise detection of the occlusal situation can be carried out. Since the tooth shape is individual and unique, biometric detection can be reliably carried out by detecting the tooth areas acting on the sensor surface.

In all of the above-mentioned methods, either the device can be enabled and used only by the intended user or, in the case of several users (possibly with different mouthpieces), the respective data can be detected, forwarded, and stored only for the detected user.

The invention therefore relates in particular to a device for optically detecting biofilm in the oral cavity, wherein at least one sensor unit is used, wherein the sensor unit has a camera that is aimed with its optical detection region into a receiving space of the device, which is positioned around surfaces that are to be detected.

According to one modification, an optical long-pass filter, which preferably has a cut-off wavelength of 480 nm to 530 nm and in particular about 510 nm and cuts off signals below this, is placed in front of the camera, in particular directly in front of it.

According to one modification, the sensor unit has at least one LED to illuminate a surface to be detected.

According to one modification, a band-pass filter or short-pass filter is placed in front of the at least one LED in order to limit/focus the wavelength spectrum of the at least one LED.

According to one modification, the at least one LED has a spectrum of 450-500 nm.

According to one modification, at least one other LED is present, which has a different spectrum, in particular a white-light spectrum.

According to one modification, a diffuser or a polarizer is positioned in front of the optics of the camera in order to avoid reflections between a protective glass and a circuit board that supports the camera and/or the at least one LED.

According to one modification, a camera base housing is positioned on the circuit board in the region of the at least one LED and for example reaches through the circuit board in the region of a through opening, wherein the camera base housing is embodied as tubular, with a first opening facing a surface to be detected and with a diametrically opposite second opening, wherein the camera base body is embodied as resting against the protective glass with an end wall that delimits the first opening.

According to one modification, the device has a handpiece and a detection attachment with an elongated cylindrical support arranged in succession, wherein a receiving region for a detection head is positioned at a free end and supports the detection head in rotary fashion.

According to one modification, the detection head is embodied in an approximately inverted U-shaped, bridge-like manner, with a base region and two legs extending from it.

According to one modification, the detection head is preferably formed in one piece and in particular, the base region and the legs are formed in one piece by means of common walls.

According to one modification, the base region and the legs are embodied as hollow and form one or more chambers for accommodating one or more sensor units and any necessary cabling, holding devices, and the like.

According to one modification, the base region and the legs define a receiving space between themselves that serves to accommodate teeth, wherein the receiving space has an opening appropriately oriented toward a tooth that is to be recorded and has an opening in each side.

According to one modification, the receiving space is delimited by essentially flat inner walls of the legs and an inner wall of the base region extending substantially orthogonal thereto, wherein the legs also have outer walls that extend from the outer edges of the inner walls delimiting the openings and transition into the outer wall of the base region, wherein the outer walls are curved so that the inner walls and outer walls form or delimit the one or more chambers.

According to one modification, facing away from the legs or facing away from the receiving space, the base region has an in particular cylindrical connection region on or in its outer wall, wherein the cylindrical connection region has a substantially round opening that is delimited by a circumferential edge, wherein a projection is embodied on the circumferential edge and forms a rotary stop.

According to one modification, on the opposite side from the cylindrical connection region, an opening is formed facing the receiving space, wherein a sensor unit is positioned in the opening inside the chamber, wherein the sensor unit is positioned in such a way that the protective glass covers the opening and the detection region of the camera correspondingly projects into the receiving space.

According to one modification, the legs have openings, which are oriented toward the receiving space, and a sensor unit is correspondingly positioned in each of them, wherein the respective panes of protective glass close the openings into the receiving space and the sensor units are positioned inside the chamber.

According to one modification, the detection regions of the sensor units in the legs point into the receiving space in diametrically opposite directions and the detection region of the sensor unit in the base region points into the receiving space orthogonally thereto and toward the opening.

According to one modification, the receiving region has a cylindrical opening which, in the mounted state, is oriented toward a row of teeth to be examined, wherein a pivot bearing is positioned in the opening in such a way that the cylindrical connection region is thus rotatably mounted in the cylindrical opening, wherein the opening of the cylindrical connection region projects into the opening.

According to one modification, the sensor units have electrical lines that are routed through the chambers of the legs and base region and through the cylindrical connection region, wherein the electrical lines serve on the one hand to connect the sensor units to a power source inside the handpiece and also to transmit acquired image data or coordinates or other data that are recorded by the cameras and that can be transmitted through these lines and either stored in the handpiece and processed or supplied for further processing via suitable transmission means, in particular a radio network, or via a conductive connection when the handpiece is inserted into a base station.

According to one modification, optionally interchangeable storage media are present in the device, in particular memory cards.

According to one modification, the electrical lines in this case are dimensioned so that the detection head can be rotated in the device without overstretching the lines.

According to one modification, spacer and guide elements are provided in order to set a sufficient distance of the cameras—particularly those in the legs—from the surface to be detected and also to ensure favorable guidance of the user when detecting the surfaces, wherein the spacer and guide elements are particularly positioned in the transition region from the inner walls to the outer walls.

According to one modification, the guide elements extend into the receiving space so that the guide elements rest against the outsides and insides of the teeth.

According to one modification, the guide elements are made of a material that is in particular biologically harmless, such as rubber or soft plastic or silicone.

According to one modification, the guide elements can be embodied as interchangeable.

According to one modification, the guide elements are positioned adjacent to the receiving space or to the outer edges of the legs on outer walls of the legs and extend either a little outward from these and then into the region of the receiving space or extend with free ends directly from the outer walls into the receiving space.

According to one modification, the optical long-pass filter, which has a cut-off wavelength of 480 nm to 530 nm and in particular about 510 nm and cuts off signals below this, is placed in front of the camera.

According to one modification, a band-pass filter or short-pass filter is placed in front of the LED in order to limit/focus the wavelength spectrum of the LED.

According to one modification, a diffuser or a polarizer is positioned between the circuit board and the protective glass.

Another aspect of the invention relates to a method for detecting biofilm in the oral cavity, wherein at least one sensor unit is used, wherein the sensor unit has a camera that is aimed with its optical detection region into a receiving space of the device, which is positioned around surfaces that are to be detected.

According to one modification, an optical long-pass filter, which preferably has a cut-off wavelength of 480 nm to 530 nm and in particular about 510 nm and cuts off signals below this, is placed directly in front of the camera.

According to one modification, at least one LED is used to illuminate a surface to be detected.

According to one modification, an image analysis comprising at least the following steps is to be performed: segmentation of the tooth surface area (of a single tooth or all visible teeth or subsections of teeth) from other oral units, in particular gums and background; analysis of the area covered with biofilm and of other oral features on the tooth surface such as de-mineralized areas, cavities, fillings, and calculus.

According to one modification, trained pattern recognition is used.

According to one modification, the recorded images undergo a manual analysis through a manual marking of the area of the image that is to be assigned to the tooth, wherein this, together with further information such as the camera position and/or tooth position, is supplied to the autonomous learning system, which preferably comprises a neural network, for learning.

According to one modification, if the autonomous learning system has been provided with enough images with corresponding additional data and markings for learning, then the system is able to mark new images of teeth independently in such a way that the tooth areas can be distinguished from non-tooth areas.

The invention will be explained by way of example based on the drawings. In the drawings:

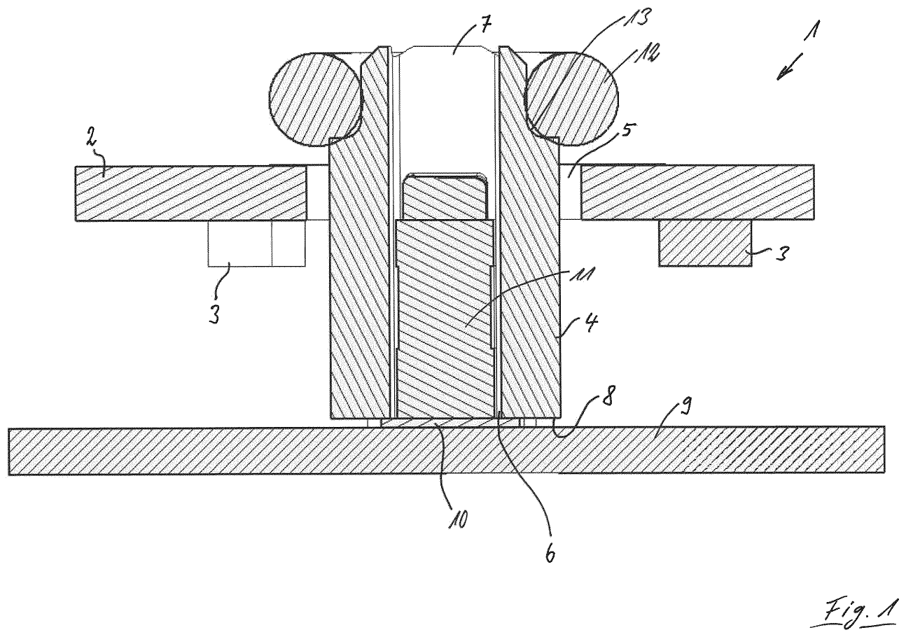

FIG. 1: shows a very schematic sectional view of a sensor unit according to the invention;

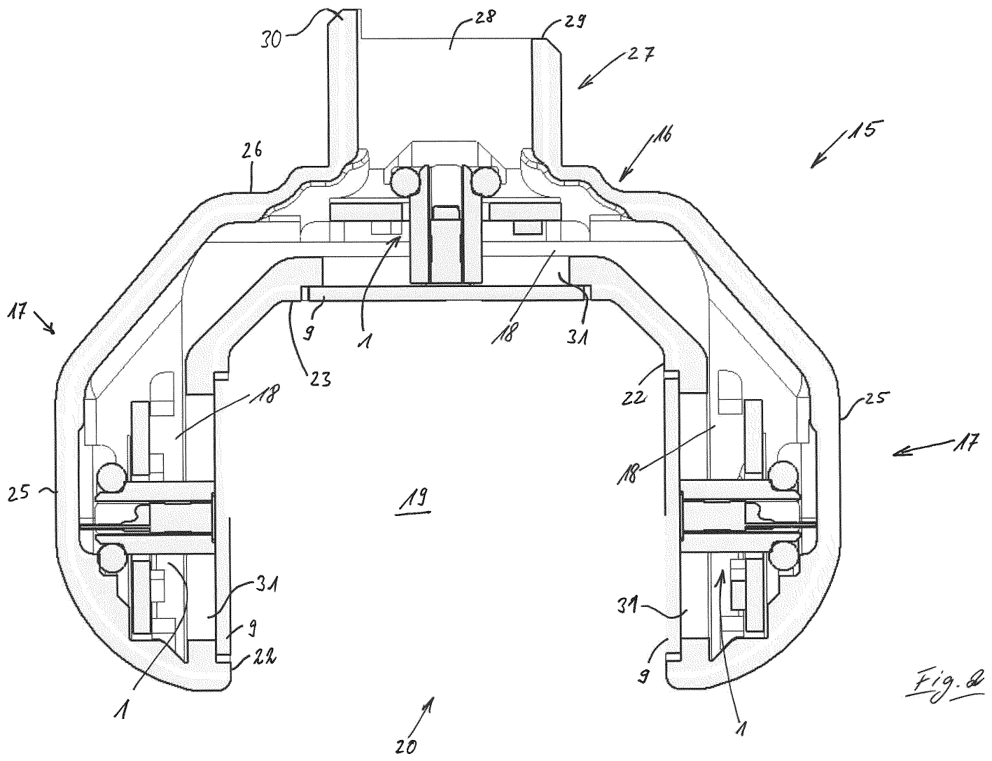

FIG. 2: shows a very schematic sectional view of a detection head according to the invention with sensor units;

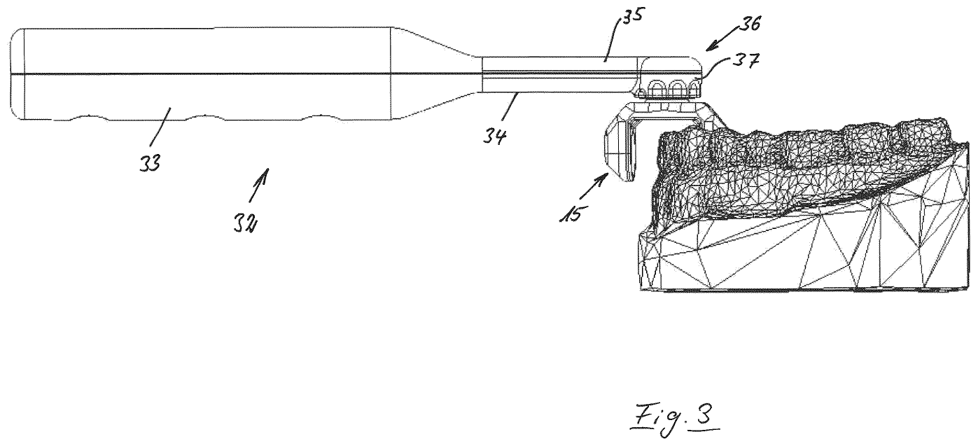

FIG. 3: shows a very schematic view of the device according to the invention with a handpiece and detection head positioned in the vicinity of incisors;

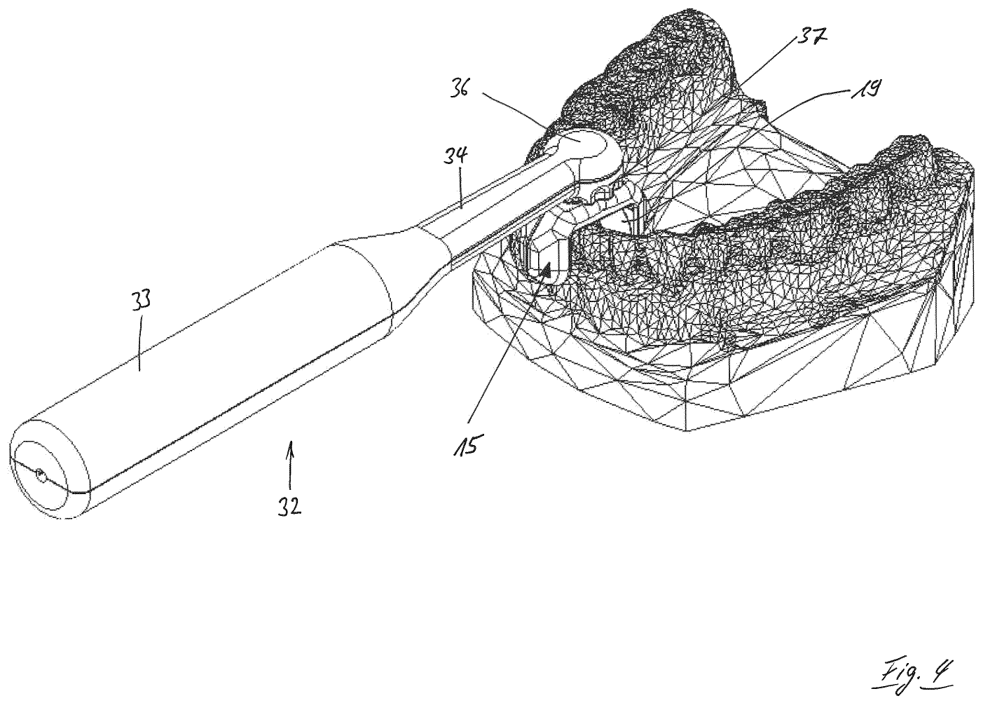

FIG. 4: shows a perspective view of the device according to FIG. 3;

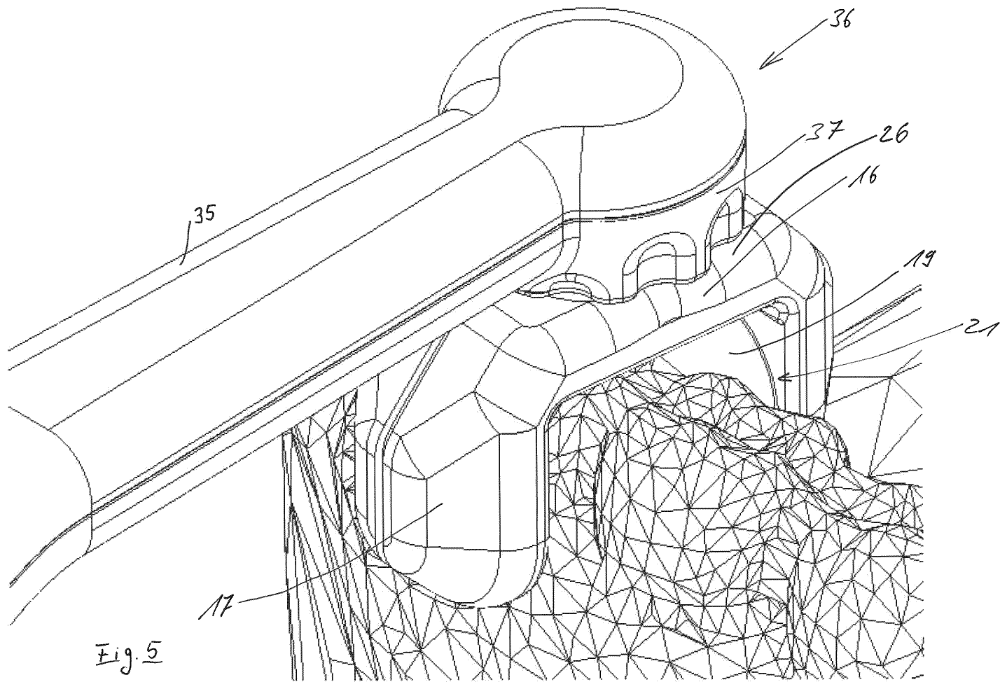

FIG. 5: shows the device according to FIG. 3 in an enlarged perspective view of the detection head;

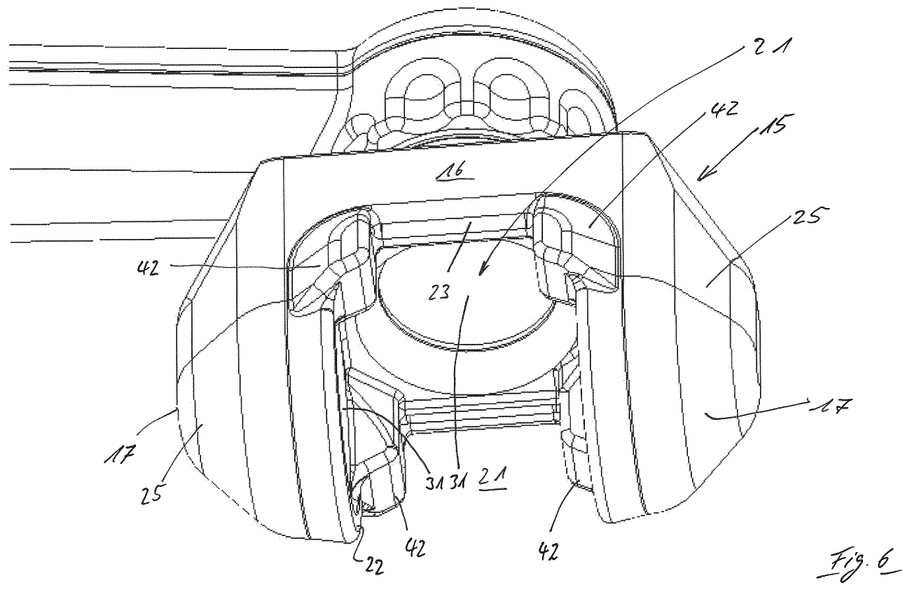

FIG. 6: shows a perspective view of one embodiment of the detection head with guide elements in a first embodiment;

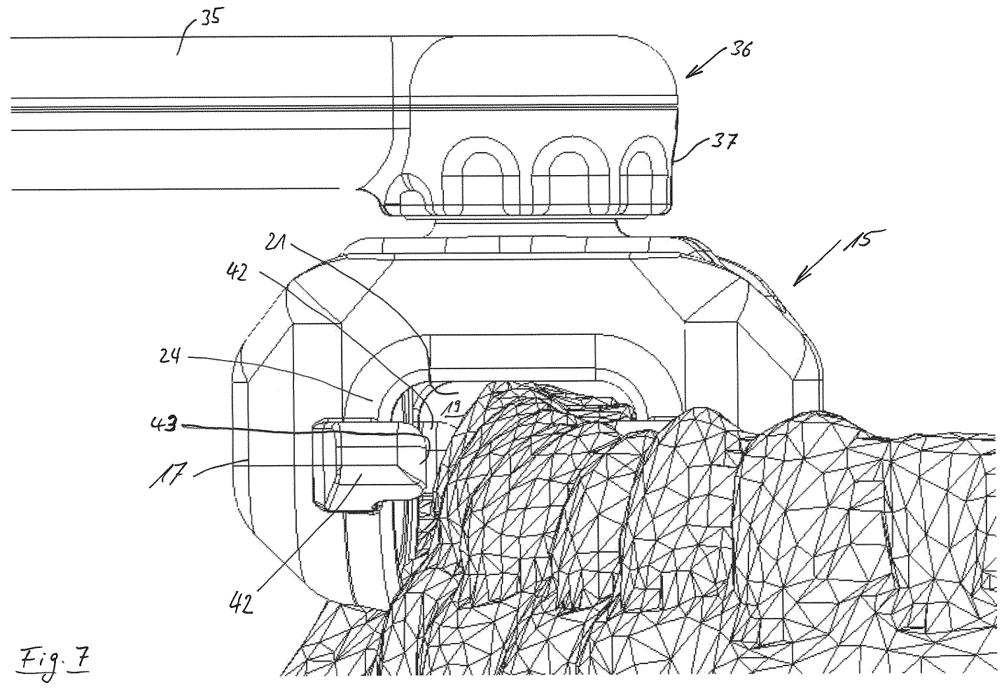

FIG. 7: shows the detection head according to FIG. 6 with guide elements in another embodiment;

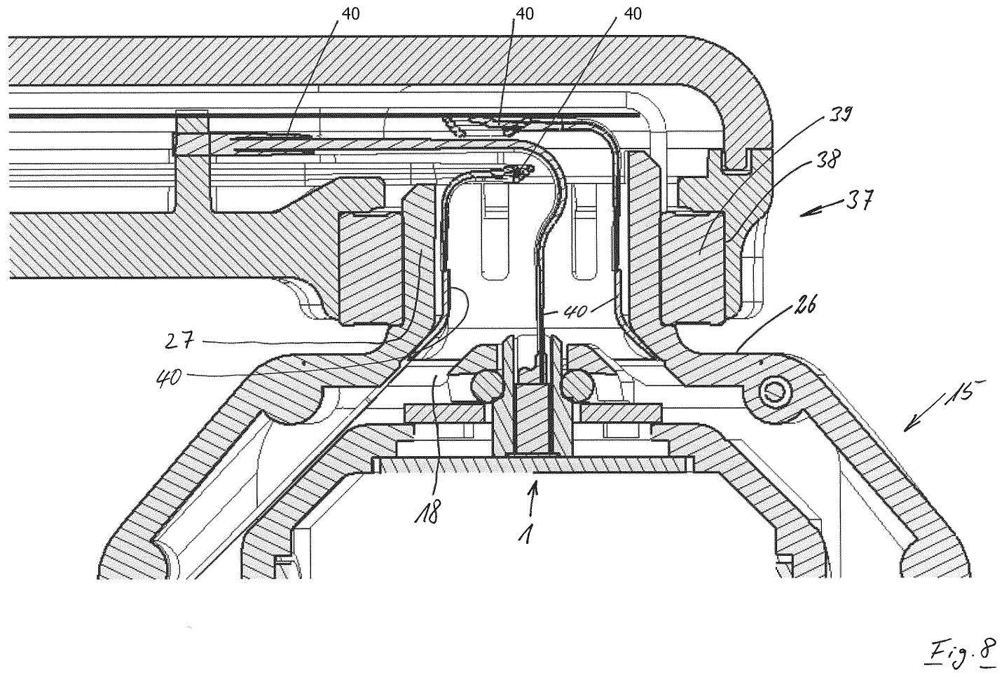

FIG. 8: shows the detection head positioned in a receiving region of the detection attachment of the device;

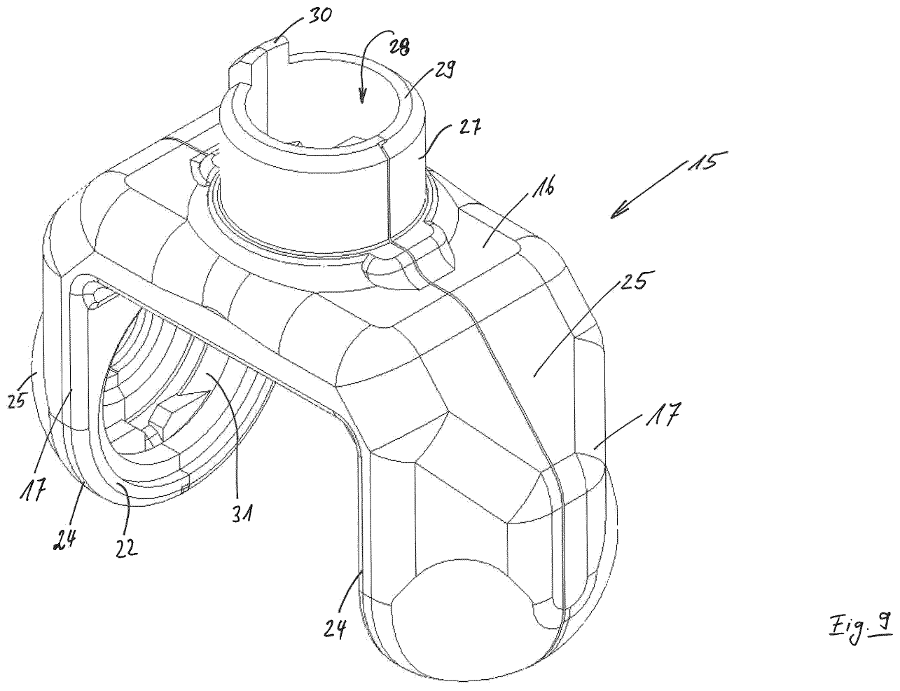

FIG. 9: shows a schematic perspective view of the detection head with its housing;

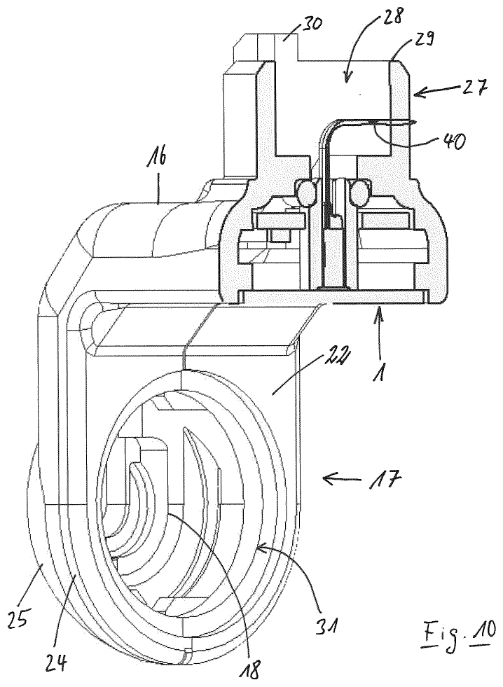

FIG. 10: shows a perspective view of a cut-away half of the detection head;

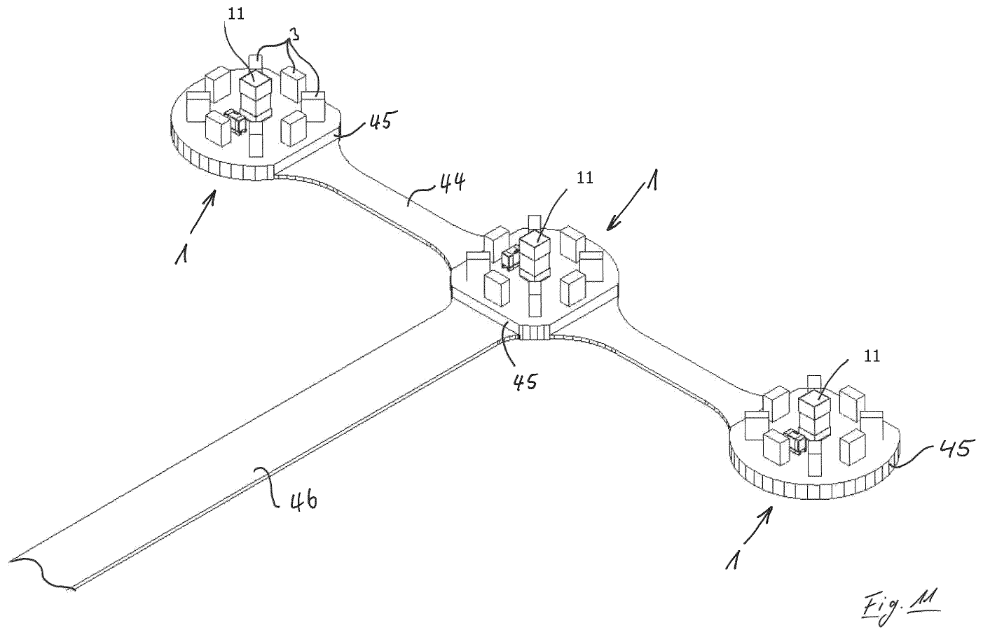

FIG. 11: shows a perspective view of another embodiment with three sensor units positioned on a flexible circuit board (PCB);

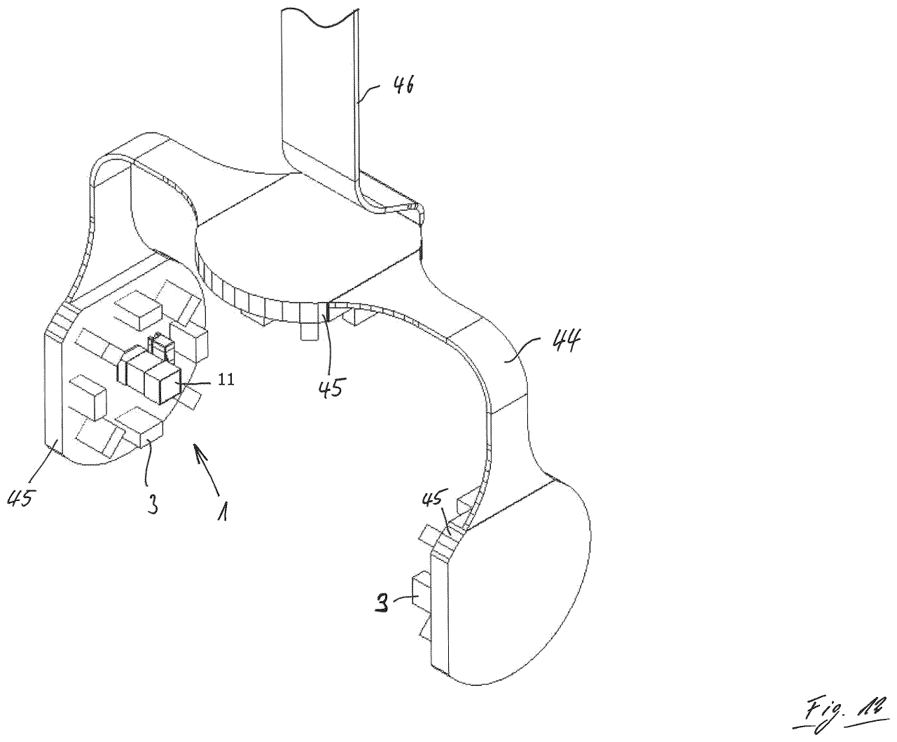

FIG. 12: shows the arrangement according to FIG. 11 in an orientation that corresponds to an installation situation;

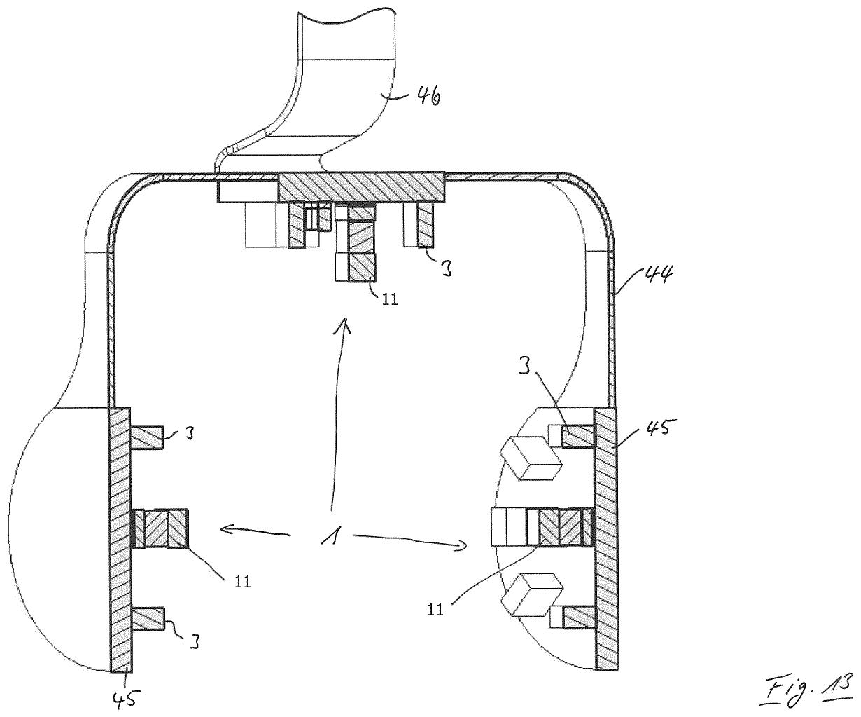

FIG. 13: shows a partially cut-away view of the arrangement according to FIG. 12;

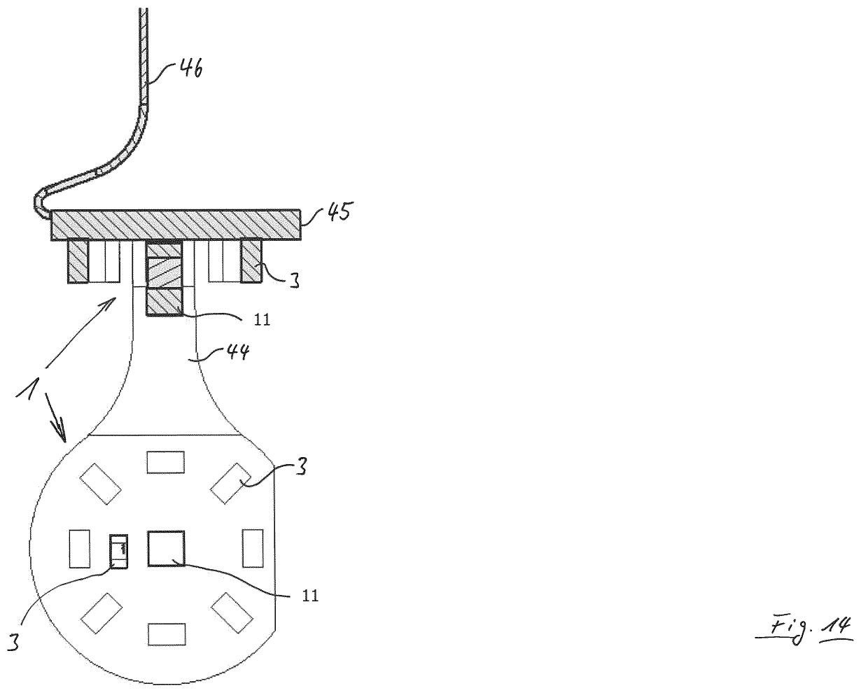

FIG. 14: shows another partially cut-away view of the arrangement according to FIG. 13.

FIG. 1 shows a very schematic sectional view of a sensor unit 1 according to the invention. The sensor unit 1 has a circuit board 2 on which at least one LED 3 is positioned in order to illuminate a surface to be detected.

A camera base housing 4 is positioned in the region of the LED 3 and for example reaches through the circuit board 2 in the region of a through opening 5. In this case, the camera base housing 4 is depicted as tubular, but it can also have any other cross-sectional shape.

The camera base housing 4 is embodied as tubular, with a first opening 6 facing an object to be inspected and with a diametrically opposite second opening 7. The camera base body 4 is embodied as resting against the protective glass 9 with an end wall 8 that delimits the first opening 6. An optical filter 10 is preferably provided between the protective glass 9 and the end wall 8.

The actual camera unit 11 is positioned inside the camera base housing 4.

For example, the camera unit 11 is a NanEye Module C or NanEye Module M camera made by AMS AG or a comparable miniaturized camera. In particular, it is a camera with a digital output, which is in particular suitable for endoscopy, for example.

For example, the camera has the dimensions 1×1×2.7 mm, as a result of which the overall sensor unit 1 has, for example, a diameter of 8 mm and a height of 3.8 mm and is therefore very small.

In particular, the camera is embodied as a wide-angle camera and can cover a field of 120°.

In the region of the opening 7 of the camera base housing 4, a circumferential seal 12 can be provide, which in particular is embodied as an O-ring and is supported by means of a corresponding circumferential groove 13.

Instead of positioning the camera unit 11 inside the camera base housing 4, the camera unit 11 can also be positioned directly against or on the circuit board 2.

FIG. 2 shows a detection head 15, which, as will be demonstrated in greater detail below, is embodied to be guided over teeth and adjacent gums in order to detect the biofilm situation. The detection head 15 is embodied in an approximately inverted U-shaped, bridge-like manner, with a base region 16 and two legs 17 extending from it.

The detection head 15 in this case is preferably formed in one piece and in particular, the base region 16 and the legs 17 are formed in one piece by means of common walls.

The base region 16 and the legs 17 are embodied as hollow and form one or more chambers 18 for accommodating one or more sensor units 1 and any necessary cabling, holding devices, and the like.

Between the regions 16, 17, the base region 16 and the legs 17 define an approximately rectangular or trapezoidal receiving space 19 that serves to accommodate teeth, wherein the receiving space 19 has an opening 20 appropriately oriented toward a tooth that is to be recorded and has an opening 21 in each side.

The receiving space 91 is delimited by essentially flat inner walls 22 of the legs 17 and an inner wall 23 of the base region 16 extending substantially orthogonal thereto.

The legs 17 also have outer walls 25 that extend from the outer edges 24 of the inner walls 22 delimiting the openings 20, 21 and transition into the outer wall 26 of the base region 16. The outer walls in this case are embodied as curved so that the inner walls 22, 23 and outer walls 25, 26 form or delimit the one or more chambers 18.

Facing away from the legs 22 or facing away from the receiving space 19, the base region 16 has an in particular cylindrical connection region 27 on or in its outer wall 26. The cylindrical connection region 27 correspondingly has a substantially round opening 28 that is delimited by a circumferential edge 29. A projection 30 is embodied on the circumferential edge 29 and forms a rotary stop, as will be explained further below.

On the opposite side from the cylindrical connection region 27, an opening 31 is formed facing the receiving space 19, wherein a sensor unit 1 is positioned in the opening 31 inside the chamber 18, wherein the sensor unit 1 is positioned in such a way that the protective glass 9 covers the opening 31 and the detection region of the camera correspondingly projects into the receiving space 19.

The legs 17 likewise have openings 31, which are oriented toward the receiving space 19, and a sensor unit 1 is correspondingly positioned in each of them, wherein the respective panes of protective glass 9 close the openings 31 into the receiving space 19 and the sensor units are positioned inside the chamber 18.

The detection regions of the sensor units 1 in the legs 17 correspondingly point into the receiving space 19 in diametrically opposite directions and the detection region of the sensor unit 1 in the base region 16 points into the receiving space 19 orthogonally thereto and toward the opening 20.

In FIG. 3 shows the entire device 32 for detecting biofilm during use. The device 32 in this case has a handpiece 33 and a detection attachment 34 with an elongated cylindrical support 35, which has a receiving region 37 at a free end 36 for the detection head 15.

In its overall appearance, the device 32 is thus similar to a conventional toothbrush so that users will be easily able to operate such a device since the feel is not unfamiliar.

The receiving region 37 in this case (FIG. 8) has a cylindrical opening 38 which, in the mounted state, is oriented toward a row of teeth to be examined, wherein a pivot bearing 39 is positioned in the opening 38 in such a way that the cylindrical connection region 27 is thus rotatably mounted in the cylindrical opening 38, wherein the opening 28 of the cylindrical connection region 27 projects into the opening 38.

The sensor units 1 have electrical lines 40 that are correspondingly routed through the chambers 18 of the legs 22 and base region 16 and through the cylindrical connection region 27 and the receiving region 37.

The electrical lines 40 serve on the one hand to connect the sensor units 1 to a power source inside the handpiece 33. In addition, acquired image data or coordinates or other data that are recorded by the camera units 11 are transmitted through these lines and are either stored in the handpiece 33 or supplied for further processing via suitable transmission means, in particular a radio network, or via a conductive connection when the handpiece is inserted into a base station.

In addition, optionally interchangeable storage media can be present in the device 32, for example memory cards, on which the corresponding data are stored so as to enable subsequent further processing of them.

The electrical lines in this case are dimensioned so that the detection head 15 can be rotated in the device 32 without overstretching the lines, wherein the projection 30 on the circumferential edge 29 of the opening 28 of the cylindrical connection region serves as a rotary stop. A possible rotation is thus limited to 320°, for example, thus making it possible to grasp the jaw branches without having to twist or pivot the handpiece 33 in an awkward way.

In particular, an ability to rotate by about 210° is already sufficient so that the detection head 15 can be correspondingly guided along the teeth in the incisor region at the start of the detection and when sweeping across the teeth (FIGS. 3-5) from the incisor region to one molar region and back to the other molar region. In order to compensate for any irregularities, a rotation of more than 180° is also possible.

Spacer and guide elements 42 can be provided in order to set a sufficient distance of the camera units 11—particularly those in the legs 17—from the surface to be detected and also to ensure favorable guidance of the user when detecting the surfaces (FIGS. 6 & 7).

The spacer and guide elements 42 are particularly positioned in the transition region from the inner walls 22, 23 to the outer walls 25, 26.

The guide elements 42 in this case can extend into the receiving space 19 and a little in the direction toward the opening 21 so that the guide elements 42 rest against the outsides and insides of the teeth.

The guide elements 42 in this case can be made of a material that is in particular biologically harmless, such as rubber or soft plastic or silicone, wherein the guide elements 42 can in particular also be embodied as interchangeable in order on the one hand, to be adaptable to individual tooth states and tooth geometries, but also in order to replace them, either when they become worn or for hygienic reasons.

In another advantageous embodiment of the guide elements 42 (FIG. 7), the guide elements 42 are positioned adjacent to the receiving space 19 or to the outer edges 24 of the legs 17 on outer walls 25 of the legs 17 and extend either a little outward from these and then into the region of the receiving space 19 or extend with free ends 43 directly from the outer walls 25 into the receiving space 19.

In order to ensure a good signal-to-noise ratio, the optical long-pass filter 10, which has a cut-off wavelength of 480 nm to 530 nm and in particular about 510 nm and cuts off signals below this, is placed directly in front of the camera unit 11. In addition (not shown) a band-pass filter or short-pass filter is placed in front of the LEDs 3 in order to limit/focus the wavelength spectrum of the LEDs. In addition to the LEDs in the range from 450 to 500 nanometers, it can also be advantageous to incorporate additional LEDs with a different spectrum.

In order to reduce reflections, which can negatively influence the recording and evaluation of the biofilm, a diffuser such as a diffuser foil or a polarizer or both can be provided between the circuit board 2 and the protective glass 9 (not shown).

A polarizer can also be placed directly in front of the camera unit 11.

The mode of operation will be explained below. The device 32, comprising the handpiece 33 and the detection attachment 34 with the detection head 15, is positioned in an oral cavity in such a way that the distance from the sensor unit 1 to the tooth surface varies between 1 and 5 mm. Detection is easily possible within this variation range.

This is aided by the corresponding guide elements 42, which may set a minimum distance or maximum distance in cooperation with each other. As already explained above, three sensor units are positioned in the detection head 15 in order to be able to detect one half of the dentition in one pass without interruption or stopping.

The detection head can also be H-shaped, i.e. doubled, so that two openings 20 are diametrically opposed. In this way, both the lower and upper half of the jaw can be accommodated at the same time.

Furthermore, instead of a device 32 with handpiece 33, it is conceivable to provide a stationary device which is positioned in stationary fashion, for example in the professional sector, i.e. at dental offices, and the patient is positioned for the purpose of establishing case histories or preparing for a professional tooth cleaning similar to an X-ray device and the stationary device then carries out the recording.

The device 32 can also be embodied without a handpiece 33 and is thus held only in the mouth.

FIGS. 11 to 14 show another embodiment according to the invention of the sensor units 1 according to the invention on a common flexible circuit board (PCB) 44. The sensor units 1 in this case are positioned directly on the common circuit board 44. In order to illuminate a surface to be detected, at least one LED 3 is respectively positioned around the camera base housing 4 or around the camera unit 11 in an SMD embodiment, which LEDs are positioned directly on the circuit board 44. In this case, the sensor units themselves can be positioned on a support structure 45 of the flexible circuit board 44 in such a way that in this region, the circuit board 44 has a sufficient rigidity for the supporting function and also can be installed well.

In this case, the camera base housing 4 is positioned on the circuit board 44 so that there is no opening. Otherwise, the embodiment of the sensor unit corresponds to the embodiment mentioned above.

A common conductor path 46 extends out from the middle sensor unit 1, for example, and thus extends (not shown) through the chamber 18 of the base region 16, the cylindrical connection region 27, and the receiving region 37.

According to the method according to the invention, the oral sensor unit is used both before and after a tooth cleaning.

Regardless of whether a measurement takes place before a tooth cleaning, for example in order to prepare for it, or after a tooth cleaning, for example to check whether this cleaning was successful, a staining must be carried out.

For this purpose, the oral biofilm in the whole oral cavity is wetted with fluorescein (or a comparable dye).

In one embodiment, this can happen in an entirely manual way in that the teeth are first rinsed with a liquid with fluorescein added to it or another transport medium for the dye (toothpaste, mouthwash, etc.).

In an alternative embodiment, a fully automatic application is possible by means of a suitable spraying, or rinsing and suction device.

With the use of a cleaning device for the oral cavity, which functions through the application of cleaning fluids, for example a device of the kind proposed by the applicant, for a measurement after the cleaning, in a last step a solution for staining the biofilm can also be introduced into the oral cavity and excess liquid can be suctioned away.

The device according to the invention is then guided over the dentition either manually or in a fully automatic way. Preferably at least 3 respective images are recorded for each tooth (inside, chewing surface, outside). In the subsequent image analysis, the quantity and location of biofilm (and other oral features) are determined per tooth.

The apparatus or device itself can also have biometric security functions. For example, a built-in camera can be used for facial recognition or a fingerprint sensor can be used for fingerprint recognition. The dental layout can also be detected using corresponding optical or mechanical pressure sensors.

Mechanical pressure sensors, for example, are a sensor field that is positioned in a mouthpiece in such a way that it points toward the crowns of the teeth when in use. In addition, the resolution—and therefore the number of sensors in the sensor field—is selected so that a very precise detection of the occlusal situation can be carried out. Since the tooth shape is individual and unique, biometric detection can be reliably carried out by detecting the tooth areas acting on the sensor surface.

In all of the above-mentioned methods, either the device can be enabled and used only by the intended user or, in the case of several users (possibly with different mouthpieces), the respective data can be detected, forwarded, and stored only for the detected user.

In particular, a standardized image of the dentition can be produced, which has a schematic image.

This information can be provided to the user in a variety of ways.

The method according to the invention in particular provides performing an image analysis comprising at least the following steps:
1. segmentation of the tooth surface area (of a single tooth or all visible teeth or subsections of teeth) from other oral units, in particular gums and background.
2. analysis of the area covered with biofilm and of other oral features on the tooth surface (de-mineralized areas, cavities, fillings, and calculus, etc.).

Primarily for the first step, special AI software solutions, in particular neural networks, are required in order to precisely distinguish the region of the tooth surface from all other oral features. This is necessary since the high signal-to-noise ratio of the sensor unit for biofilm results in the fact that other oral units are less easily distinguished and conventional methods such as threshold calculation or spectral analysis only function insufficiently.

For the second step, thresholds or spectral analyses are able to distinguish very well between the different features. A refined AI software solution, however, can significantly increase the precision and reduce the required computing power.

In principle, a threshold analysis for biofilm is used in order to distinguish regions that are covered with biofilm from tooth regions that are not covered.

Preferably, trained pattern recognition is used in this case.

For this purpose, the recorded images undergo a manual analysis through a manual marking of the area of the image that is to be assigned to the tooth. This, together with further information such as the camera position and/or tooth position, is supplied to the autonomous learning system, which preferably comprises a neural network, for learning.

If the autonomous learning system has been provided with enough images with corresponding additional data and markings for learning, then the system is able to mark new images of teeth independently in such a way that the tooth areas can be distinguished from non-tooth areas.

The invention has the advantage that a method and device are created for detecting biofilm in the oral cavity in a simple and understandable way and data are thus provided, which enable a targeted cleaning by means of corresponding cleaning devices.

In particular, image files and/or coordinate files are generated, which can be further processed in a cleaning device for purposes of positioning and/or controlling the intensity and/or controlling the duration of a cleaning.

### REFERENCE NUMERAL LIST

1 sensor unit
2 circuit board
3 LED
4 camera base housing
5 through opening
6 first opening
7 second opening
8 end wall
9 protective glass
10 optical filter
11 camera unit
12 circumferential seal
13 groove
15 detection head
16 base region
17 legs
18 chamber(s)
19 receiving space
20 opening
21 side openings
22 inner walls
23 inner wall
24 outer edges
25 outer walls
26 outer wall
27 cylindrical connection region
28 opening
29 circumferential edge
30 projection serving as rotary stop
31 opening
32 device
33 handpiece
34 detection attachment
35 elongated cylindrical support
36 free end
37 receiving region
38 cylindrical opening
39 pivot bearing
40 connecting cable
42 spacer and guide elements
43 free ends
44 flexible circuit board
45 support structure
46 common conductor path

1. A device (32) for optically detecting biofilm in the oral cavity, wherein at least one sensor unit (1) is provided, wherein the sensor unit (1) has a camera unit (11), which points with its optical detection region into a receiving space (19) of the device (32) and serves to record surfaces that are to be detected, wherein an LED (3), a circuit board (2), and a protective glass (9) are provided in the device, wherein
an optical long-pass filter (10), which has a cut-off wavelength of 480 nm to 530 nm and cuts off signals below this, is placed in front of the camera unit (11) or a diffuser or a polarizer is positioned between the circuit board (2) and the protective glass (9) or the polarizer and diffuser are both present; or
the optical long-pass filter (10) is placed in front of the camera unit (11) and a diffuser or a polarizer is positioned between the circuit board (2) and the protective glass (9) or the polarizer and diffuser are both present.

2. The device according to claim 1, characterized in that the optical long-pass filter (10) is placed directly in front of the camera unit (11).

3. The device according to claim 1, characterized in that the sensor unit (1) has at least one LED (3) configured to illuminate a surface to be detected.

4. The device according to claim 1, characterized in that a band-pass filter or short-pass filter is placed in front of the at least one LED (3) in order to limit/focus the wavelength spectrum of the at least one LED (3).

5. The device according to claim 3, characterized in that the at least one LED has a spectrum of 450-500 nm.

6. The device according to claim 5, characterized in that at least one other LED (3) is present, which has a different spectrum.

7. The device according to claim 1, characterized in that a diffuser or a polarizer is positioned in front of the optics of the camera unit (11) and configured to avoid reflections between a protective glass (9) and a circuit board (2) that supports the camera unit (11) and/or the at least one LED (3).

8. The device according to claim 1, characterized in that a camera base housing (4) is positioned on the circuit board (2) in a region of the at least one LED (3) and reaches through the circuit board (2) in a region of a through opening (5), wherein the camera base housing (4) is embodied as tubular, with a first opening (6) facing a surface to be detected and with a diametrically opposite second opening (7), wherein the camera base body (4) is embodied as resting against the protective glass (9) with an end wall (8) that delimits the first opening (6).

9. The device according to claim 1, characterized in that the device (32) comprises a handpiece (33) and a detection attachment (34) with an elongated cylindrical support (35) arranged in succession, wherein a receiving region (37) for a detection head (15) is positioned at a free end (36) and supports the detection head in rotary fashion.

10. The device according to claim 2, characterized in that the detection head (15) is embodied in an approximately inverted U-shaped, bridge-like manner, with a base region (16) and two legs (17) extending from it.

11. The device according to claim 10, characterized in that the detection head (15) is formed in one piece and in particular, the base region (16) and the legs (17) are formed in one piece by means of common walls (22, 23, 25, 26).

12. The device according to claim 10, characterized in that the base region (16) and the legs (17) are embodied as hollow and form one or more chambers (18) for accommodating one or more sensor units (1).

13. The device according to claim 10, characterized in that the base region (16) and the legs (17) define a receiving space (19) between themselves that serves to accommodate teeth, wherein the receiving space (19) has an opening (20) appropriately oriented toward a tooth that is to be recorded and has an opening (21) in each side.

14. The device according to claim 13, characterized in that the receiving space (19) is delimited by inner walls (22) of the legs (17) and an inner wall (23) of the base region (16) extending at an angle thereto, wherein the legs (17) also have outer walls (25) that extend from outer edges (24) of the inner walls (22) delimiting the openings (20, 21) and transition into an outer wall (26) of the base region (16), wherein the outer wall (26) is curved so that the inner walls (22, 23) and outer walls (25, 26) form or delimit the one or more chambers (18).

15. The device according to claim 10, characterized in that facing away from the legs (17) or facing away from the receiving space (19), the base region (16) has a cylindrical connection region (27) on or in its outer wall (26), wherein the cylindrical connection region (27) has a substantially round opening (28) that is delimited by a circumferential edge (29), wherein a projection (30) is embodied on the circumferential edge (29) and forms a rotary stop.

16. The device according to claim 15, characterized in that on an opposite side from the cylindrical connection region (27), an opening (31) is formed facing the receiving space (19), wherein a sensor unit (1) is positioned in the opening (31) inside the chamber (18), wherein the sensor unit (1) is positioned in such a way that the protective glass (9) covers the opening (31) and a detection region of the camera correspondingly projects into the receiving space (19).

17. The device according to claim 14, characterized in that the legs (17) have openings (31), which are oriented toward the receiving space (19), and a sensor unit (1) is correspondingly positioned in each of them, wherein respective panes of protective glass (9) close the openings (31) into the receiving space (19) and the sensor units are positioned inside the chamber (18).

18. The device according to claim 17, characterized in that detection regions of the sensor units (1) in the legs (17) point into the receiving space (19) in opposite directions and the detection region of the sensor unit (1) in the base region (16) points into the receiving space (19) orthogonally thereto and toward the opening (20).

19. The device according to claim 15, characterized in that the receiving region (37) has a cylindrical opening (38) which, in a mounted state, is oriented toward a row of teeth to be examined, wherein a pivot bearing (39) is positioned in the opening (38) in such a way that the cylindrical connection region (27) is thus rotatably mounted in the cylindrical opening (38), wherein the opening (28) of the cylindrical connection region (27) projects into the opening (38).

20. The device according to claim 17, characterized in that the sensor units (1) have electrical lines (40) that are routed through the chambers (18) of the legs (17) and base region (16) and through a cylindrical connection region (27), wherein the electrical lines (40) are configured to connect the sensor units (1) to a power source inside the handpiece (33) and also to transmit acquired image data or coordinates or other data that are recorded by the cameras (11) and that can be transmitted through these lines and either stored in the handpiece (33) or supplied for further processing via transmission means or via a conductive connection when the handpiece is inserted into a base station.

21. The device according to claim 1, characterized in that optionally interchangeable storage media are present in the device (32).

22. The device according to claim 20, characterized in that the electrical lines (40) are dimensioned so that it is possible to rotate the detection head (15) in the device (32) without overstretching the lines.

23. The device according to claim 17, characterized in that spacer and guide elements (42) are provided in order to set a sufficient distance of the sensor units (1) in the legs (17) from the surface to be detected and also to ensure favorable guidance of the user when detecting the surfaces, wherein the spacer and guide elements (42) are particularly positioned in the transition region from the inner walls (22, 23) to the outer walls (25, 26).

24. The device according to claim 23, characterized in that the guide elements (42) extend into the receiving space (19) so that the guide elements (42) are configured to rest against the outsides and insides of tooth surfaces to be detected.

25. The device according to claim 23, characterized in that the guide elements (42) are made of a material that is biologically harmless.

26. The device according to claim 23, characterized in that the guide elements (42) are interchangeable.

27. The device according to claim 23, characterized in that the guide elements (42) are positioned adjacent to the receiving space (19) or to the outer edges (24) of the legs (17) on outer walls (25) of the legs (17) and extend either a little outward from these and then into the region of the receiving space (19) or extend with free ends (43) directly from the outer walls (25) into the receiving space (19).

28. The device according to claim 1, characterized in that a band-pass filter or short-pass filter is placed in front of the LED (3) and configured to limit/focus the wavelength spectrum of the LED.

29. A method for detecting biofilm in the oral cavity, comprising:
obtaining a device according to claim 1; and
using the device to detect a surface in the oral cavity.

30. The method according to claim 29, characterized in that at least one LED (3) is used to illuminate the surface to be detected.

31. The method according to claim 29, characterized in that an image analysis comprising at least the following steps is to be performed: segmentation of a tooth surface area (of a single tooth or all visible teeth or subsections of teeth) from other oral units, in particular gums and background; analysis of the area to determine the extent, if any, the area is covered with biofilm and analysis of at least one other oral feature on the tooth surface.

32. The method according to claim 31, characterized in that trained pattern recognition is used.

33. The method according to claim 32, characterized in that recorded images of the area undergo a manual analysis through a manual marking of the area of the image that is to be assigned to the tooth, and wherein the manual analysis is supplied to an autonomous learning system for learning.

34. The method according to claim 33, characterized in that if the autonomous learning system has been provided with enough images with corresponding additional data and markings for learning, then the system is able to mark new images of teeth independently in such a way that the tooth areas can be distinguished from non-tooth areas.
